(19) United States
(12) Patent Application Publication
(10) Pub. No.: US 2021/0106684 A1
(43) Pub. Date: Apr. 15, 2021

(54) NANO-SILICA PARTICLES CONTAINING BORON ISOTOPE AND SERVING AS BORON NEUTRON CAPTURE AGENT

(71) Applicant: DYNAMIC SCIENCE CORPORATION, Chuo-ku (JP)

(72) Inventors: Hitoshi HORI, Chuo-ku (JP); Ken TOKUNAGA, Chuo-ku (JP); Masao TAKAHASHI, Chuo-ku (JP)

(73) Assignee: DYNAMIC SCIENCE CORPORATION, Chuo-ku (JP)

(21) Appl. No.: 16/982,172

(22) PCT Filed: Sep. 19, 2018
(86) PCT No.: PCT/JP2018/035823

§ 371 Date: Dec. 21, 2020

(30) Foreign Application Priority Data
Mar. 19, 2018 (JP) 2018-072734

(51) Int. Cl. A61K 41/00; A61K 47/69; A61K 47/64; A61K 47/54; A61K 31/69; A61K 47/26

(52) U.S. Cl. CPC A61K 41/0095; A61K 47/6923; A61K 47/26; A61K 47/542; A61K 31/69; A61K 47/64

(57) ABSTRACT

The present invention is the creation of stable boron isotope (¹⁰B) containing nanosilica particles composing of amino acids, amino-oligosaccharides, and oligopeptides appropriate to the improvement of drug delivery system (DDS) function of delivering more boron to tumors selectively to facilitate the efficacy of boron neutron capture therapy (BNCT).

### FIELD OF THE INVENTION

The present invention relates to a drug material specialized in drug delivery system (DDS), for boron neutron capture therapy (BNCT), comprising a stable nanosilica particles and a drug that enables specific accumulation in cancer cells in BNCT by using amino acids, amino-oligosaccharides and oligopeptides, the drug being chemically bonded to the nanosilica particles.

### BACKGROUND OF THE INVENTION

Boron neutron capture therapy (BNCT) is a therapeutic modality of treating the tumor using alpha particles generated form the nuclear reaction of boron isotope (¹⁰B) and thermal neutron beam to yield the destruction of tumor cells. It is very important that a large number of boron isotope (¹⁰B) atoms have to be delivered and localized within cancer cells and malignant tumor cells for BNCT to be effective.

Theoretically, the selectively accumulated boron isotope (¹⁰B) compounds can kill cancer cells and malignant tumor cells without any damage of surrounding normal cells to yield successful BNCT. However, the currently used BNCT drugs, such as mercaptoundecahydrododecaborate (BSH) and p-borono-phenylalanine (p-BPA), are not optimal boron isotope (¹⁰B) compounds for BNCT: BSH does not have membrane permeability enough to reach in tumor cells and p-BPA (intravenous drip at a large amounts of 20-30 g p-BPA/60 kg bodyweight) needs excess amounts (1:1 stoichiometry) of fructose or polyols to improve its extremely poor water solubility, being not patient friendly. An approach to deliver and accumulate boron isotope (¹⁰B) atoms to cancer cells using boron compounds encapsulated a combination of polyethyleneglycol and an envelope vector was reported (Patent document 1), with difficulty of being practically used. Another boron delivery system using boron-containing cell membrane-permeable peptides (Patent document 2), was reported to be administered intravenously in the form of conventional capsules, gels, and emulsions as DDS excipient carriers. The boron dose of 0.05-1.0 g/kg body weight necessary to secure the therapeutically effective intracellular accumulation amount of 10-25 ppm, which is the similar dose used conventionally being not patient-friendly. The situation of progress in boron isotope DDS suggests that both p-BPA and BSH drugs are difficult to adapt as BNCT drugs for all cancer treatment.

The present invention is first accomplished from the diligent study on tumor amino acid transporter (Non-patent document 3) which comes to highly express in cancer cells and tumor cells, particularly focusing on four kinds of proteins such as LACT1, LAT3, ATBO^{0,+}, and ASCT2 that apt to express specifically in tumor cells and their broad substrate selectivity. In view of this, an oligopeptide highly relating to transported substrate amino acid is synthesized (claim 5), the oligopeptide sequence being inserted to the nanosilica particles which can be connected with a boron isotope (¹⁰B) thereby to make it possible to provide an oligopeptide-boron isotope (¹⁰B) nanosilica particles.

On the other hand, in cancer cells and tumor cells, aerobic glycolysis is developed and the amount of glucose is increased under Warburg effect, thereby highly enhancing an expression of a glucose transporter. Taking advantage of this phenomenon, the next invention is the creation of amino-oligosaccharide-boron isotope (¹⁰B) nanosilica particles containing glucosamine, mannosamine, galactosamine, and neuraminic acid as glucose-mimetic substrates appropriate to Nat-glucose transporter, which nanosilica particles are to be delivered to cancer cells an tumor cells.

Thus there is no boron isotope (¹⁰B) compound that exhibits good selective accumulation property and accumulation concentration at the depth of cancer cells and malignant tumor cells and exhibits the selective accumulation property and a drug delivery property also in administration to a patient.

### PRIOR ART LITERATURES

### Patent Documents

Patent document1: JP-A-2008-308440

Patent document2: JP-A-2013-87098

### Non-Patent Documents

Non-patent document 1: Am. J. Cancer Res. (2005) 5(4): 1281-1294.

Non-patent document2: Proc. Natl. Acad. Sci. USA (1976); 73: 396-400.

Non-patent document3: Pharmacol. Ther. (2009); 121:29-40.

### SUMMARY OF THE INVENTION

### Problems to be Solved by the Invention

The present invention was accomplished to solve the above-mentioned problems, and provides a drug for boron neutron capture therapy (BNCT) containing stable nanosilica particles as matrix and a drug delivery material (DDS) having specific accumulation property in cancer cells and tumor cells, the drug being obtained by chemically bonding nanosilica particles to a boron isotope (¹⁰B)-containing compound comprising amino-oligosaccharides, substrate amino acids and oligopeptides.

### Means to Solve Problems

Porous spherical silica particles used in the present invention are spherical nanosilica particles with a diameter of 5 nm to 2000 nm and a surface area of 100-300 m²/g. Reactive functional groups are first chemically modified on this large surface area of nanosilica particles. Into the modified groups on the nanosilica particles, boron isotope (¹⁰B) molecular moiety or fragment is introduced, followed by the chemical reaction of oligosaccharides, substrate amino acids, and oligopeptides, thereby to produce the present invented a boron isotope (¹⁰B)-containing compound.

As the first invention, a basic structure of the present invention, i.e., a boron isotope (¹⁰B)-containing nanosilica particles (compound 1) is shown in the formula (1):

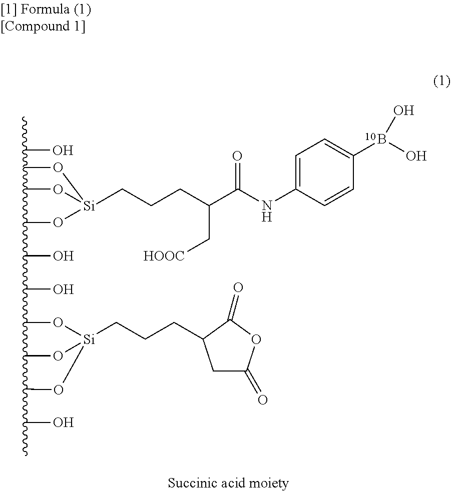

wherein the above-mentioned oligosaccharides, substrate amino acids, and oligopeptides are to be bonded chemically to the succinic acid moiety thereby producing a BNCT drug for various tumor cells.

The second invention is shown as follows.

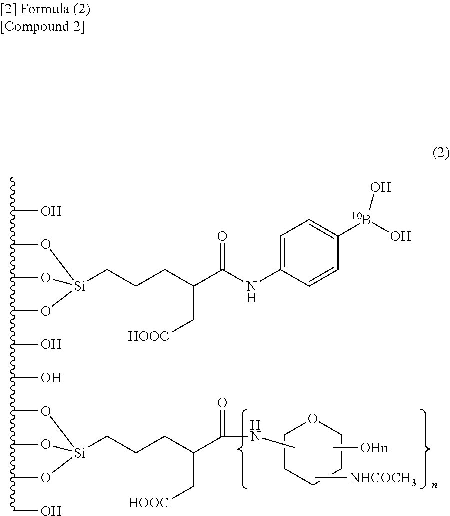

The compound 2 according to the formula (2) is a compound obtained by chemically bonding oligoglucosamine to the succinic acid moiety of the formula (1). This compound is provided as a drug delivery agent having an affinity to polysaccharides known as a glycocalyx of cancer cells.

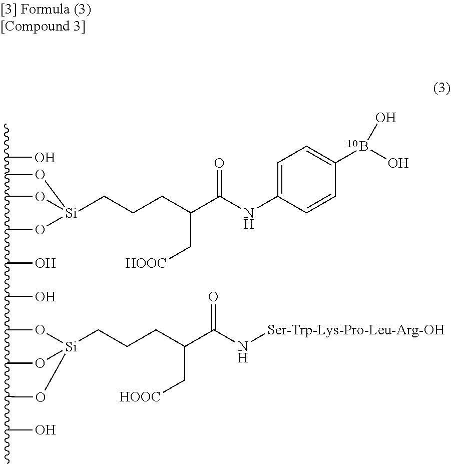

The compound 3 according to the formula (3) is a compound wherein an oligopeptide Ser-Trp-Lys-Pro-Leu-Arg, a representative example of oligopeptides, is chemically bonded to the succinic acid moiety in the formula (1). This compound provides a BNCT drug serving as a ligand specifically bondable to specific tumor cells.

### Effects of the Invention

The invented oligopeptide modified boron isotope (¹⁰B) containing nanosilica particles are effectively delivered to and selectively localized or accumulated in the depth core inside cancer cells and tumor cells at a high boron isotope (¹⁰B) concentration. The invented oligosaccharide modified boron isotope (¹⁰B)-containing nanosilica particles are selectively delivered and localized around the cell membrance of tumor cells to promote extremely high cytotoxic effects on tumor cells under neutron irradiation.

### BRIEF DESCRIPTION OF DRAWINGS

FIG. 1 shows a structural formula of a basic compound of the present invention, i.e., an aminophenylboronic acid isotope (¹⁰B)-connected nanosilica particles modified with succinic anhydride.

### MODE FOR CARRYING OUT THE INVENTION

Porous spherical nanosilica particles used in the present invention as a drug delivery system (DDS) material have a particle diameter of from 5 nm to 2000 nm, preferably from 5 nm to 50 nm, but is not particularly limited.

The nanosilica particles can be administrated intravenously through a neovascularized coarse blood wall closer to the tumor tissue to accumulate into the tumor cells at high boron isotope (¹⁰B) concentration. Additionally, the nanosilica used in the present invention has a large surface area of 100-300 m²/g so as to be able to chemically bond oligosaccharides, substrate amino acids, and oligopeptides thereto at high concentrations. The typical examples are shown as follows.

### EXAMPLE 1

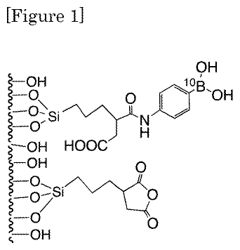

Synthesis of a basic compound of the present invention, i.e., an aminophenylboronic acid (boron isotope (¹⁰B))-connected nanosilica particles modified with succinic anhydride (Compound 1) as shown in FIG. 1

For the synthesis of succinic anhydride-modified nanosilica particles, nanosilica SiO₂ (10.0 g; particle size of 10 nm, but not limited to this particle size) was washed with acetone (100 mL), then washed with isopropanol (100 mL), and finally washed with deionized water, and dried in the microwave and then dried under nitrogen stream. To the resulted nanosilica particles, 2-[3-triethoxysilylpropyl] succinic anhydride (0.3-0.9 mol; its molar concentration is not particularly limited) dissolved in ethanol (100 mL) was added. The reaction mixture was heated at 120° C. for 1.5 hours. The resulted reaction mixture was washed with isopropanol (100-150 mL) and dried under vacuum to yield the desired succinic anhydride-modified nanosilica particles. Subsequently, the obtained succinic anhydride-modified nanosilica particles (2.0 g) was added to prepare an aqueous solution (10 ml) and then a boron isotope (¹⁰B)-containing 4-aminophenylboronic acid (2-200 mg; more preferably 40-120 mg) was added thereto. The reaction temperature was set at 0-60° C. (more preferably 20-30° C., but it is not particularly limited). Thus an aminophenylboronic acid (boron isotope (¹⁰B)-connected and succinic anhydride-modified nanosilica particles (as shown in FIG. 1) was obtained at 70-80 yield.

### EXAMPLE 2

Synthesis of aminophenylboronic acid (boron isotope (¹⁰B))-connected oligosaccharide nanosilica particles

### (Typical Example)

Oligoglucosamine (5-100 mmoL) was dissolved in 10 ml of water, and 1.0 g of the compound obtained in the example 1 was added thereto. The reaction was carried out at 5-40° C. for 0.5-120 min (the conditions of temperature and time are not particularly limited). The resulted mixture was filtered and then stirred for 5-120 min with the addition of 10 mL of HEPES (4-(2-hydroxyethyl)-1-piperazineethanesulfonic acid) buffer solution. The mixture was filtered and then washed with ethanol, thereby obtaining the desired compound.

### EXAMPLE 3

Synthesis of aminophenylboronic acid (boron isotope (¹⁰B))-connected oligopeptide nanosilica particles

### (Typical Example)

An oligopeptide having a sequence of Ser-Trp-Lys-Pro-Leu-Arg (3-10 mg) was suspended in MES buffer (10 mL) and then stirred with the addition of 0.1 g of the compound obtained in the example 1 to carried out a reaction for 0.5-120 min (more preferably 1-30 min, but is not particularly limited) at 3-60° C. The reaction mixture was washed with an excess amount of water and then washed with an excess amount of acetone and finally dried under vacuum, thereby to obtain the aminophenylboronic acid (boron isotope (¹⁰B)-connected oligopeptide nanosilica particles.

1-6. (canceled)

7. A compound specialized in Drug Delivery System (DDS), for use in “boron-neutron capture therapy (BNCT)” the compound being synthesized such that an amino acid and an oligopeptide are bonded to boron isotope (¹⁰B) using a reactive functional group with which porous spherical nanosilica particles having a particle diameter of from 5 to 2000 nm are chemically modified.

8. A compound or drug specialized in DDS, for use in BNCT, obtained by causing transportable substrates of amino acid transporters that are highly expressed in cancer cells to chemically bond to the compound of claim 7 thereby synthesizing an amino acid boron isotope (¹⁰B).

9. A method for synthesizing nanosilica particles to which only phenylboronic acid (boron isotope (¹⁰B)) is chemically bonded, and a drug or compound utilizing the same for BNCT, provided that boron isotope (¹¹B) is not contained.

10. A method for synthesizing oligosaccharide-constituting silica particles that comprise phenylboronic acid (boron isotope (¹⁰B)).

11. A compound or synthesis method, wherein the constituent oligopeptide relating to claim 7 is a cell membrane-permeable oligopeptide having a sequence of:
Ser-Leu-Ile-Val-Met-Thr-Cys-Arg,
Ser-Les-Pro-Thr-,
Tyr-Tyr-Arg-Ala-Tyr,
Ser-Trp-Lys-Pro-Leu-Arg,
or
Tyr-Ser-Lys-Cys-His.

12. The method for synthesizing boron isotope (¹⁰B) nanosilica particles compound, as claimed in claim 10, wherein the oligosaccharide comprises, as constituent amino-saccharides, glucosamine, mannosamine, galactosamine, and neuraminic acid.
